(19) United States
(12) Patent Application Publication
(10) Pub. No.: US 2020/0281876 A1
(43) Pub. Date: Sep. 10, 2020

(54) Topical Preparation for the Treatment of Arthritis and Skin Conditions

(71) Applicant: Alvin J. MARX, San Antonio, TX (US)

(72) Inventor: Alvin J. MARX, San Antonio, TX (US)

(21) Appl. No.: 16/296,029

(22) Filed: Mar. 7, 2019

(51) Int. Cl. A61K 31/138; A61K 31/522; A61K 33/06; A61K 31/404; A61K 31/573; A61K 9/06; A61K 9/00; A61P 19/02; A61P 17/00; A61K 9/70

(52) U.S. Cl. CPC A61K 31/138; A61K 31/522; A61K 33/06; A61K 31/404; A61K 9/70; A61K 9/06; A61K 9/0014; A61P 19/02; A61P 17/00; A61K 31/573

(57) ABSTRACT

A composition, a method of manufacture, and a method of application for the treatment of arthritis, skeleton-muscular joint inflammation, and certain skin conditions. The composition comprises a mixture of diphenhydramine, caffeine, magnesium, and petrolatum mixed to form an ointment for application to the affected part of the body. The method of application includes the use of a wet porous membrane, such as a wet paper towel, covering the applied ointment on the skin for a period of time after application.

### BACKGROUND OF THE INVENTION

### 1. Field of the Invention

The present invention relates generally to topical ointments for the treatment of joint and soft tissue ailments as well as skin conditions. The present invention relates more specifically to a topical preparation for the treatment of arthritis, tendonitis and other joint and soft tissue conditions. The present invention is also useful for skin conditions such as lesions, irritations, inflammations, and burns.

### 2. Description of the Related Art

The novelty of the present invention lies in the combination of ingredients, the method of compounding, and the method of use. Each of the primary constituent ingredients has been used separately in topical formulations to treat conditions unrelated or remotely related to the object conditions treated by the present invention.

### BACKGROUND ON THE PRIMARY CONSTITUENTS

The ingredients in this topical preparation have been chosen in part because all have been used orally and topically with minimum side effects. One goal of the present invention is to have a preparation that is safe enough to not require FDA approval before manufacturing and distribution.

Antihistamine—

Antihistamines are drugs that oppose the action of histamines. The preferred antihistamine in the preparation of the present invention is diphenhydramine commonly sold OTC under the brand name Benadryl®, which blocks H-1 histamine receptors. Stimulation of these receptors increases vascular permeability to fluid and inflammatory agents, and promotes angiogenesis, thereby further increasing inflammation. The antihistamine in this preparation decreases histamine effects thereby decreasing swelling, inflammation and pain. Diphenhydramine has been used for more than sixty years. It is usually given orally and is also used as a local anesthetic. The concentration in this preparation is 0.5%, with a range of 0.025-4.0%.

Further Background on Diphenhydramine—

Diphenhydramine is an antihistamine mainly used to treat allergies. It is also used for insomnia, symptoms of the common cold, tremor in Parkinson's disease, and nausea. It is used primarily by mouth, and also by intramuscular or intravenous injection. Topical uses of diphenhydramine are well established. Maximal effect for the oral dose is typically around two hours after a dose, and effects can last for up to seven hours. Common side effects include sedation, poor coordination, and gastritis. Its use is not recommended in babies. There is no clear risk of harm when used during pregnancy; however, use during breastfeeding is not recommended. It is a first generation H-1 antihistamine and works by blocking certain effects of histamine. Diphenhydramine is also an anticholinergic.

Xanthine Derivatives—

Xanthine derivatives are thought to act by causing ATP (adenosine triphosphate) to go to cAMP (cyclic adenosine monophosphate). The cAMP inhibits degranulation of inflammatory cells such as basophils, eosinophils and most specifically mast cells. The preferred embodiment of the present invention uses caffeine, a methylated xanthine and active ingredient in coffee. In its pure form caffeine is a white powder. About half of the OTC pain medications contain caffeine. Thousands of papers have been written about caffeine and it is considered to be safe in proper amounts. In excess amounts, caffeine can cause both hypertension and hypotension. The preferred concentration in the preparation of the present invention is 0.75% with a range of 0.3-3.0%.

Further Background on Caffeine—

Caffeine is a central nervous system (CNS) stimulant of the methylxanthine class. It is the world's most widely consumed psychoactive drug. Unlike many other psychoactive substances, it is legal and unregulated in nearly all parts of the world. There are several known mechanisms of action to explain the effects of caffeine. The most prominent is that it reversibly blocks the action of adenosine on its receptor and consequently prevents the onset of drowsiness induced by adenosine. Caffeine also stimulates certain portions of the autonomic nervous system. Caffeine is a bitter, white crystalline purine, a methylxanthine alkaloid, and is chemically related to the adenine and guanine bases of deoxyribonucleic acid (DNA) and ribonucleic acid (RNA). It is found in the seeds, nuts, or leaves of a number of plants native to Africa, East Asia and South America, and helps to protect them against predator insects and to prevent germination of nearby seeds. The most well-known source of caffeine is the coffee bean. Beverages containing caffeine are ingested to relieve or prevent drowsiness and to improve performance. To make these drinks, caffeine is extracted by steeping the plant product in water, a process called infusion. Caffeine containing drinks, such as coffee, tea, and cola, are very popular. As of 2014, 85% of American adults consumed some form of caffeine daily, consuming 164 mg daily on average. Caffeine can have both positive and negative health effects. It can treat and prevent asthma as well as premature infant breathing disorders. Caffeine citrate is on the WHO Model List of Essential Medicines. It may confer a modest protective effect against some diseases, including Parkinson's disease. Some people experience sleep disruption or anxiety if they consume caffeine, but others show little disturbance. Evidence of a risk during pregnancy is equivocal; some authorities recommend that pregnant women limit consumption to the equivalent of two cups of coffee per day or less. Caffeine can produce a mild form of drug dependence. When an individual stops using caffeine after repeated daily intake, withdrawal symptoms develop including sleepiness, headache, and irritability. Tolerance to the autonomic effects of increased blood pressure and heart rate, and increased urine output, develops with chronic use. Caffeine is classified by the U.S. Food and Drug Administration as generally recognized as safe (GRAS). Toxic doses, over 10 grams per day for an adult, are much higher than the typical dose of under 500 milligrams per day. A cup of coffee contains 80-200 mg of caffeine, depending on cup size, what type of coffee bean is used, and how it is prepared. Thus it requires roughly 50-100 ordinary cups of coffee to reach the toxic dose. However, pure powdered caffeine, which is available as a dietary supplement, can be lethal in tablespoon amounts.

Magnesium—

Magnesium is important in many cellular functions. It is a catalyst that is necessary for ATP to go to ADP, the basic pathway of cellular energy production by the Krebs cycle. Magnesium sulfate is the magnesium salt used in the preferred embodiment of the present invention. Magnesium has been used for years as a bath soak for muscle pain. The concentration of magnesium in the present invention is 20 g per 100 g of ointment with a range of 2 g-40 g per 100 g of ointment.

Further Background on Magnesium—

Magnesium sulfate is an inorganic salt with the formula MgSO₄(H₂O)ₓ where x equals 0 to 7. It is often encountered as the heptahydrate sulfate mineral epsomite (MgSO₄.7H₂O), commonly called Epsom Salt. The overall global annual usage in the mid-1970s of the monohydrate was 2.3 million tons, of which the majority was used in agriculture. Epsom Salt has been traditionally used as a component of bath salts. Epsom Salt can also be used as a beauty product. Athletes use it to soothe sore muscles, while gardeners use it to improve crops.

Compound Transport (Ointment)—

The preferred embodiment for the means of application and transport of the efficacious components of the present invention is in an ointment. The preferred vehicle of the present invention is petrolatum (commonly sold OTC under the brand name Vaseline®). Petrolatum is known to help in the healing process of skin damage. This vehicle should therefore be considered an active ingredient. The exact mechanism of action is not fully understood. Originally, Vaseline® was developed for treatment of skin irritations. Other medicament transport vehicles such as gels, oils or creams are acceptable.

Further Background on Petrolatum—

Petroleum jelly, petrolatum, white petrolatum, soft paraffin, or multi-hydrocarbon, CAS number 8009-03-8, is a semi-solid mixture of hydrocarbons (with carbon numbers mainly higher than 25), originally promoted as a topical ointment for its healing properties. After petroleum jelly became a medicine chest staple, consumers began to use it for many conditions, including toenail fungus, genital rashes (non-STD), nosebleeds, diaper rash, and chest colds, as well as in cosmetic preparations. Its folkloric medicinal value as a “cure-all” has since been limited by better scientific understanding of appropriate and inappropriate uses. It is recognized by the U.S. Food and Drug Administration (FDA) as an approved over-the-counter (OTC) skin protectant and remains widely used in cosmetic skin care.

Wetting Agent—

Facilitating the process of compounding the formulation of the present invention is the use of a wetting agent. Because the preference is to avoid the spoilage that can occur with water based compositions, glycerine and/or mineral oil are preferred in the present invention.

Further Background on Glycerine—

Glycerol (also called glycerine or glycerin) is a simple polyol compound. It is a colorless, odorless, viscous liquid that is sweet-tasting and non-toxic. The glycerol backbone is found in many lipids which are known as glycerides. It is widely used in the food industry as a sweetener and humectant and in pharmaceutical formulations. Glycerol has three hydroxyl groups that are responsible for its solubility in water and its hygroscopic nature. Glycerine is the preferred wetting agent in the present invention due to its ability to rapidly dissolve the active ingredients in the formulation process and due to the preferred tactile character it provides to the formulation. Glycerine is a colorless, odorless, syrupy, sweet liquid with a formula of C₃H₈O₃. It is manufactured by saponification of natural fats and oils. It has been used for sweetening and preserving food. It is also used in the manufacturing of cosmetics, perfumes, inks, glues and cement. As a solvent it can be used in automobile antifreeze, medical suppositories and skin emollients. It was first used in 1838. It is generally regarded as safe (GRAS) by the FDA.

Background on Mineral Oil (Alternative Wetting Agent)—

Mineral oil is any of various colorless, odorless, light mixtures of higher alkanes from a mineral source, particularly a distillate of petroleum. The name mineral oil by itself is imprecise, having been used for many specific oils over the past few centuries. Other names, similarly imprecise, include white oil, paraffin oil, liquid paraffin (a highly refined medical grade), paraffinum liquidum (Latin), and liquid petroleum. Most often, mineral oil is a liquid by-product of refining crude oil to make gasoline and other petroleum products. This type of mineral oil is a transparent, colorless oil, composed mainly of alkanes and cycloalkanes, related to petroleum jelly. It has a density of around 0.8 g/cm³.

Other Possible Active Ingredients—

Other ingredients in the formulation of the present invention may include aspirin (acetylsalicylic acid); NSAIDs (non-steroidal anti-inflammatory drugs); acetaminophen (commonly sold OTC under the brand name Tylenol; other beta-2 agonist (such as albuterol); and glucocortosteroids.

Other Possible Inactive Ingredients—

Alcohol and ammonia may be added to help in absorption of medication into and through the skin.

### SUMMARY OF THE INVENTION

The purpose of the pharmaceutical preparation of the present invention is to provide analgesia for local pain and to facilitate healing. The active ingredients include an antihistamine, a xanthine derivative, a magnesium compound and petrolatum. For joint and soft tissue conditions, the basic formulation is designed to treat conditions that include osteoarthritis, other types of arthritis, arthralgia, tendonitis, and fibromyalgia. For such conditions, the ointment should be applied over and around the joint or other painful area. For skin conditions, the ointment can be used to treat conditions that include acne, surgical skin wounds, and superficial burns. The ointment should be applied to the skin over and around the active lesion/surgical wound. If this preparation is used over more than 10% of the skin area, some of the dosage limits and/or riders would have to be reduced or substituted for. This preparation is designed for short term use of up to 3 weeks.

### BRIEF DESCRIPTION OF THE DRAWINGS

FIG. 1 is a flowchart diagram disclosing a first preferred method of compounding the ointment formulation of the present invention.

FIG. 2 is a flowchart diagram disclosing the method of use steps in a representative process for application of the ointment formulation of the present invention.

FIG. 3 is a schematic diagram of the human knee joint (as a representative skeleto-muscular joint) showing application of the ointment formulation of the present invention.

### DETAILED DESCRIPTION OF THE PREFERRED EMBODIMENTS

All of the medications and components in the ointment of the present invention have been used in topical preparations. The benefit of this preparation is that all of the active agents are conveniently compounded into a single preparation that exhibits synergistic effects on the conditions being treated. Initial tests and patient use show efficacy for the reduction of joint and skin discomfort beyond what might be expected through the use of the individual ingredients alone or in separate application. As indicated above, while each of the active ingredients used in the present invention have been used in topical preparations, such is not their most common mechanism for use or application.

### First Formulation Composition (Example I)

Diphenhydramine Hydrochloride USP 0.5 g per 100 g of ointment.

Caffeine Anhydrous USP 0.75 g per 100 g of ointment.

Magnesium Sulfate Heptahydrate USP 20 g per 100 g of ointment.

Petrolatum White USP 63.75 g per 100 g of ointment.

Glycerine USP 15 g per 100 g of Ointment.

For Total Composition of 100 g.

It is worth noting that since the preparation of the present invention may be spread over a wide skin area, the concentrations of components are lower than those in other topical preparations. When used in oral medications, typical dosages include: diphenhydramine 25 mg, caffeine 200 mg, and magnesium 400 mg.

### First Formulation Compounding (Example I)

(a) Identify and weigh quantities of components.

(b) Place all ingredients into an appropriate size container.

(c) Wet with enough glycerine to dissolve dry ingredients.

(d) Add required amount of petrolatum.

(e) Mix (spin on medium setting until homogenous)

The preferred order and method of mixing the active ingredients is to add the powder of diphenhydramine, and the ground powders of magnesium and caffeine crystals to the specified quantity of warmed glycerine. This process facilitates the rapid dissolving of the dry ingredients.

### Second Formulation Composition (Example II)

Diphenhydramine Hydrochloride USP 0.5 g per 100 g of ointment.

Caffeine Anhydrous USP 0.75 g per 100 g of ointment.

Magnesium Sulfate Heptahydrate USP 20 g per 100 g of ointment.

Petrolatum White USP 63.75 g per 100 g of ointment.

Mineral Oil USP to wet.

To make a Total Composition of 100 g of ointment.

### Second Formulation Compounding (Example II)

(a) Identify and weigh quantities of components.

(b) Grind magnesium sulfate crystals and caffeine anhydrous crystals to a powder.

(c) Weigh out all ingredients into an appropriate size container.

(d) Wet with enough mineral oil to make a paste.

(e) Add required amount of petrolatum.

(f) Mix (spin on medium setting until homogenous)

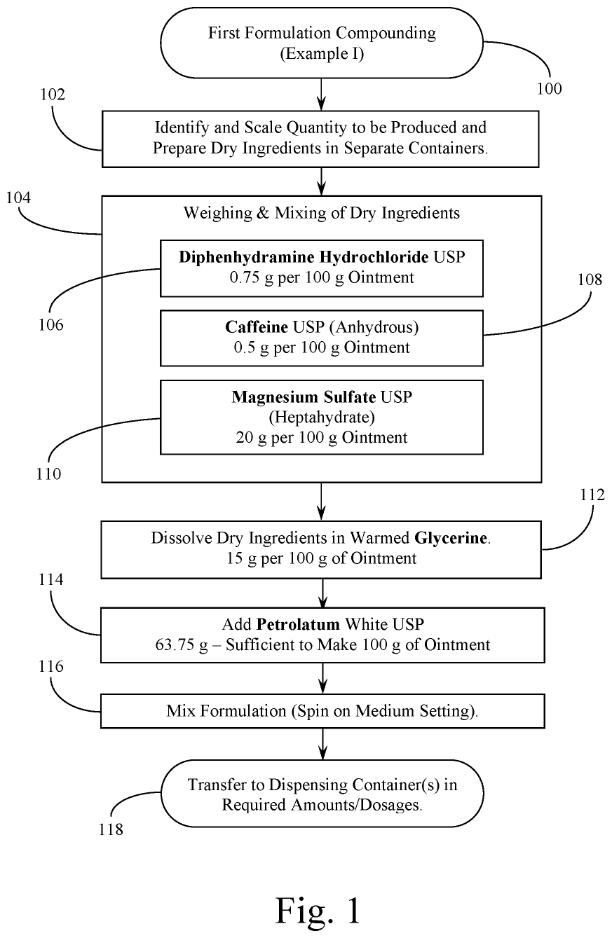

Reference is made to FIG. 1 for a summary of the steps for compounding the ointment formulation of the present invention. The first formulation compounding is initiated at Step 100 and reflects the preferred use of glycerine as the wetting agent. Step 102 sets forth the process of calculating the component quantities based upon a selected batch size. While there are no specific limitations on batch size, the examples provided in the preferred embodiment result in a formulation having active ingredients totaling 100 g per batch.

Step 104 includes the process of weighing and mixing the primary active ingredients; Step 106, the diphenhydramine hydrochloride component; Step 108, the caffeine component; and Step 110, the magnesium sulfate component. The mixture of dry ingredients resulting from Step 104 is then mixed into a quantity of glycerine at Step 112 to fully dissolve the ingredients in a brief (1-4 minutes) period of time at room temperature. Step 114 involves the addition of petrolatum to the glycerine wetted mixture. The resultant compound is placed into a mixing container for a spin mixer and is spin mixed at Step 116 on a medium setting for an appropriate period of time (generally less than four minutes). Finally the formulated ointment is transferred, at Step 118, to an appropriately sized and configured dispensing container in the required amounts/dosages.

Use & Topical Application

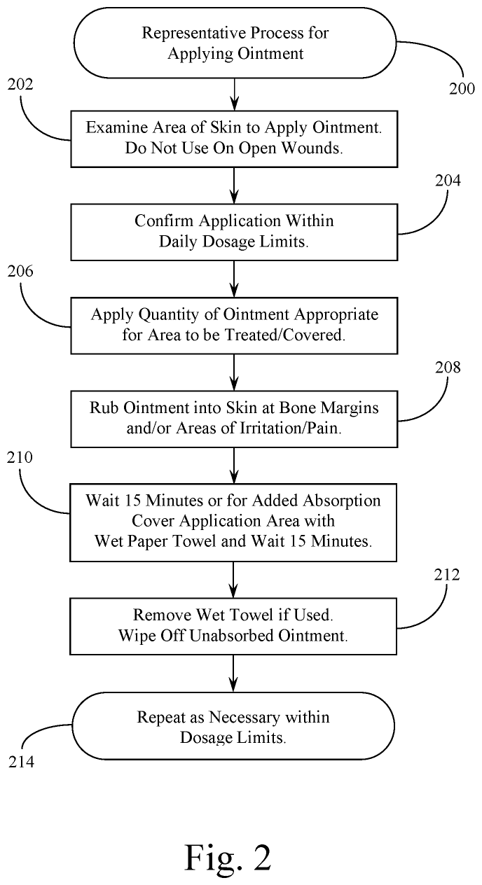

Reference is next made to FIG. 2 for a flowchart diagram disclosing the method steps in a representative process for application of the ointment formulation of the present invention. The representative process for applying the ointment is initiated at Step 200 and begins with the user examining the area of skin to which the ointment is to be applied at Step 202. Although the ointment is suitable for the treatment of skin irritations and lesions, like most other topical ointments it should not be used on open wounds. The next pre-application requirement is carried out at Step 204 with the user confirming that the proposed application does not exceed the recommended daily dosage limits. As indicated above, it would be difficult to exceed dosage limitations of 10% of body area, with a topical application of the active ingredients although some restrictions and limitations are still advised.

Step 206 involves the actual dispensing of the ointment from the container in a quantity appropriate for the area to be treated. Such dispensing may be made in incremental amounts over the area to be treated and then followed at Step 208 with the process of rubbing the dispensed quantities into the skin. Depending on the purpose of the application the ointment should be rubbed into the skin at the bone margins associated with a painful joint or directly onto the irritated area of skin. Step 210 involves the added absorption by placing a wet paper towel over the area of application to facilitate the transport of the active ingredients into the skin over a brief (15 minute) period of time. The process of placing a moist barrier on the skin after application of the ointment serves to insure that a greater portion of the active ingredients are transported through the layers of skin.

At Step 212, if used, the wet paper towel is removed. Wipe off any unabsorbed ointment from the application area. The removal of the excess ointment is primarily a matter of user comfort and generally does not bear upon the dosage limitations or requirements. If the pain or irritation is not relieved at the application site, the process may be repeated at Step 214, again within the daily dosage limitations set forth for the formulation.

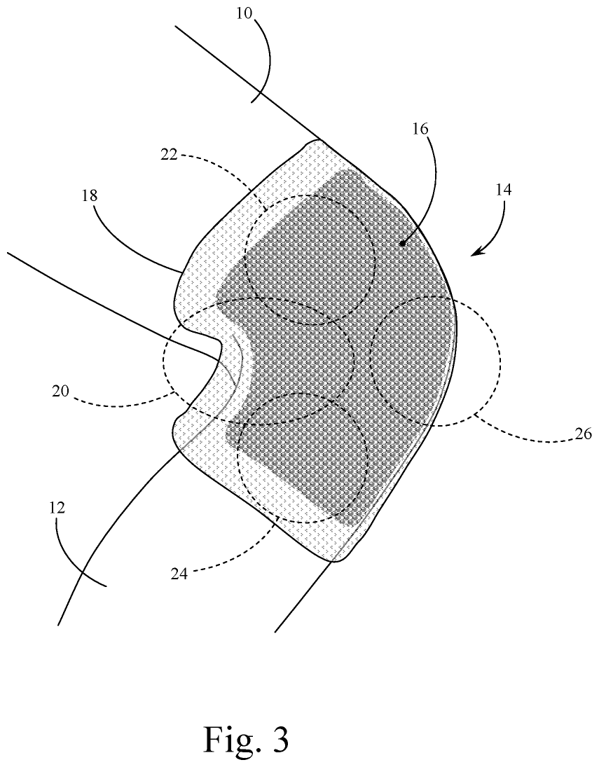

FIG. 3 is a schematic diagram of the human knee joint (as a representative skeleto-muscular joint) showing implementation and application of the ointment formulation of the present invention. In humans and other primates, the knee joins the thigh 10 with the leg 12 and consists of two joints: one between the femur and tibia (tibiofemoral joint), and one between the femur and patella or kneecap 14 (patellofemoral joint). The knee is the largest joint in the human body. The knee is a modified hinge joint, which permits flexion and extension as well as slight internal and external rotation. The knee is vulnerable to injury and to the development of osteoarthritis.

The areas of application of the ointment of the present invention are shown schematically in FIG. 3 and provide some guidance for the user to target effective areas to address pain and discomfort in the joint. Application of the ointment 16 liberally around the entire joint is appropriate. As indicated above, a wet paper towel 18 should be placed over the area of ointment application and left in place for approximately 15 minutes. The properties of the wet paper towel generally insure that it will remain in place, generally intact, to facilitate the transport of the ointment, already rubbed into the skin, into the irritated or inflamed tissues of the joint.

The knee joint, like most joints, is a complex assembly of bones, muscles, cartilage, tendons, ligaments, bursae, blood vessels, and synovial tissues. Any or all of these tissue structures can become inflamed and present a source of pain or discomfort. The goal of the ointment formulation of the present invention is to be transported into the inflamed or irritated tissues by the most direct path. Because of the lack of significant side effects, however, there is little need to limit the application of the ointment to a perceived source of pain. Overall application, as shown in FIG. 3, remains appropriate where the source of the pain is not clear. Nonetheless, it is worth focusing on the areas of the joint where the internal structures of the joint that are most likely inflamed are located. For example, side area 20 in the region of the tibiofemoral joint at the “bone margins” would be an important application region. Likewise, upper joint area 22 and lower joint area 24, in the region where muscles and tendons are connected into the joint target significant sources of inflammation pain. Finally, with the knee, the patellofemoral joint (highlighted by joint area 26) just underneath the kneecap, can also be a source of inflammation pain that is directly accessible below the skin for the ointment of the present invention to be effective. Similar areas for optimal application may be identified for other joints in the human body.

Osteoarthritis Applications—

Osteoarthritis (OA) is the most common type of arthritis. The knee joint is the most common joint affected. Osteoarthritis is also commonly seen in the hands, wrists, feet, shoulders, ankles. hips, and spine. It is characterized by joint swelling, pain and deformity of cartilage and bone, accompanied by inflammation of the synovium (joint capsule). Osteoarthritis is generally treated with physical therapy, exercise, anti-inflammatory drugs and weight loss. Systemic anti-inflammatory drugs can be used when necessary. OA affects about 30-50 million people in the United States. It is associated with injury, age and gender.

Use for Treatment of Arthritis—

As discussed above, the ointment should be applied over and around the painful joint(s) one to two times daily. The ointment should not be applied to more than four joints at one time. The ointment should not be used on areas where the skin is damaged. Use of the ointment should be discontinued after three weeks. The ointment should not be used if the patient is allergic to any of the ingredients. Use should be discontinued if a skin rash or other allergic symptoms occur.

Use for Treatment of Skin Conditions—

The ointment should be applied in a thin coat once or twice a day. The ointment should not be used over more than 10% of the total surface area of the body at any one time. Use of the ointment should be discontinued after three weeks. The ointment should not be used if the patient is allergic to any of the ingredients. Use should be discontinued if the condition gets worse, a new rash occurs, or after three weeks.

Advantages of Topical Preparations—

Topical medication is applied locally to affected areas in places of pain, skin inflammation, or irritation. The topical preparation of the present invention may be applied liberally to the skin over and around the arthritic joint or the area of adverse skin condition. Systemic absorption of topical medication can be as much as 60%. The four primary ingredients in this topical preparation combine to help reduce inflammation. The topical preparation can deliver higher doses of medication directly to the region of the joint with minimal systemic side effects.

The goal of moving to a topical approach for the alleviation of pain and inflammation is in part beneficial as a manner of reducing dependency on the use of opioids. The current opioid overdose epidemic involves over 70,000 deaths a year in the United States due to excess systemic use. Many addicts become addicted to opioids after being started on them by their physicians. NSAIDS are a non-opioid group of pain medications whose side effects include arteriosclerotic cardiovascular disease. Patients still, however, need to treat persistent joint pain to prevent a chronic pain syndrome and loss of function. Safe non-opioid based medications are needed. The ingredients in the topical preparation of the present invention have been chosen because all have been used systemically (and to a lesser degree topically) with minimal side effects.

The ointment of the present invention works by slowing and reducing the inflammation process with diphenhydramine and caffeine, and speeding the healing process with magnesium and petrolatum. The ointment does not contain water and therefore does not require preservatives as may be necessary with many skin creams. Preservatives can cause skin irritation and allergies. Petrolatum melts at skin temperature which makes it further advantageous as a base carrier and active ingredient of the composition of the present invention. The combination of ingredients is softer and easier to apply than petrolatum alone.

Although the present invention has been described in connection with a number of preferred embodiments, those skilled in the art will recognize that modification of the formulation (quantities and ratios of the active ingredients) may be made without departing from the spirit and scope of the invention. While the primary active ingredients are essential to the efficacy of the formulation, a variety of carriers other than glycerine and mineral oil might be anticipated. In addition, other mechanisms that achieve the same functionality as a wet paper towel cover over the ointment application site for a period of time might also be anticipated. Further, ancillary compounds, as mentioned above, might be added to the formulation to act independently or synergistically in a beneficial manner to treat the pain or discomfort in the joint or on the skin. Such ancillary components may simply take advantage of the dermal transport mechanism provide by the formulation of the present invention as long as there are no adverse effects or adverse interactions with the ingredients of the present formulation.

I claim:

1. A composition for treating arthritis characterized by joint inflammation and swelling in a mammalian subject, the composition comprising a therapeutically effective amount of diphenhydramine; a therapeutically effective amount of caffeine; and a therapeutically effective amount of magnesium, the therapeutically effective amount in each case being sufficient to reduce joint inflammation, pain and swelling.

2. The composition of claim 1 wherein the therapeutically effective amount of diphenhydramine is in the form of diphenhydramine hydrochloride.

3. The composition of claim 2 wherein the therapeutically effective amount of diphenhydramine hydrochloride is in the range of 0.25 g to 4 g per 100 g of composition.

4. The composition of claim 1 wherein the therapeutically effective amount of caffeine in the form of anhydrous caffeine.

5. The composition of claim 4 wherein the therapeutically effective amount of anhydrous caffeine is in the range of 0.3 g to 3 g per 100 g of composition.

6. The composition of claim 1 wherein the therapeutically effective amount of magnesium is in the form of magnesium sulfate heptahydrate.

7. The composition of claim 6 wherein the therapeutically effective amount of magnesium sulfate heptahydrate is in the range of 2 g to 40 g per 100 g of composition.

8. The composition of claim 1 further comprising a therapeutically effective amount of petrolatum.

9. The composition of claim 8 wherein the therapeutically effective amount of petrolatum is in the form of petrolatum white.

10. A method for treating arthritis characterized by joint inflammation and swelling in a mammalian subject with the topical application of a compounded ointment, the ointment composition comprising a therapeutically effective amount of diphenhydramine; a therapeutically effective amount of caffeine; a therapeutically effective amount of magnesium, and a therapeutically effective amount of petrolatum, the therapeutically effective amount in each case being sufficient to reduce joint inflammation, pain and swelling.

11. The method of claim 10, wherein the arthritis comprises one or more conditions selected from the group consisting of osteoarthritis, rheumatoid arthritis, psoriatic arthritis and inflammation resulting from any of these conditions.

12. The method of claim 10, wherein the compounded ointment further comprises a non-steroidal anti-inflammatory drug.

13. The method of claim 10, wherein the compounded ointment further comprises a glucocorticoid.

14. The method of claim 10 wherein the topical application of the ointment is made on the skin in an area over and/or proximal or distal to a skeleton-muscular joint.

15. The method of claim 14 wherein the topical application of the ointment comprising rubbing a quantity of the ointment into the skin around and/or over the joint.

16. The method of claim 15 further comprising the step of covering the ointment application area with a flexible wet membrane for a period of time to facilitate absorption of the active ingredients into the affected joint tissues.

17. The method of claim 16 wherein the flexible wet membrane comprises a wet paper towel sized to cover and generally adhere to the area of application of the ointment.

18. The method of claim 16 further comprising the step of, after the period of time, removing the flexible wet porous membrane with any excess, unabsorbed ointment from the surface of the skin.

19. The method of claim 10 further comprising the step of repeating the topical application of the ointment on an area over or proximal to a skeleton-muscular joint up to a maximum daily dosage for the active ingredients in the ointment.

20. The method of claim 10 wherein the compounded ointment results in a composition having a tactile application characteristic making the ointment easier to apply to the skin than petrolatum alone.
